(19) United States
(12) Patent Application Publication
(10) Pub. No.: US 2010/0330664 A1
(43) Pub. Date: Dec. 30, 2010

(54) Treatment System Utilizing a Fluid Replacement Cartridge

(75) Inventor: Barnet Liberman, New York, NY (US)

Correspondence Address: COHEN, PONTANI, LIEBERMAN & PAVANE LLP, 551 FIFTH AVENUE, SUITE 1210, NEW YORK, NY, 10176, US

(73) Assignee: Winterlab Limited, New York, NY (US)

(21) Appl. No.: 12/746,405

(22) PCT Filed: Dec. 8, 2008
(86) PCT No.: PCT/US08/13473

§ 371 Date: Sep. 3, 2010

Related U.S. Application Data
(60) Provisional application No. 61/005,596, filed on Dec. 6, 2007.

(51) Int. Cl. C12M 1/00

(52) U.S. Cl. 435/307.1

(57) ABSTRACT

A treatment system, in particular a system using a brine solution for chilling or freezing tissue samples, includes a working vessel wherein samples can be treated, and a replacement cartridge having a first compartment with an inlet, and a second compartment with an outlet, wherein the second compartment is collapsible. A first conduit connects the working vessel to the inlet; and a second conduit connects the outlet to the working vessel. Used working fluid can be moved from the tank to the first compartment via the first conduit, and fresh working in the second compartment can be forced into the second conduit as the second compartment collapses. A holding vessel can be provided in the first conduit for holding used fluid, or in the second conduit for holding the fresh fluid, while the working vessel is cleaned.

### BACKGROUND

1. Technical Field

The present invention relates to a treatment system having a working vessel with a working fluid for treatment of samples, and more particularly to a tissue sample freezing or chilling system using a brine solution.

2. Description of the Related Art

A treatment system having a treatment vessel containing a brine solution used to lower the temperature of samples immersed in the solution is already known. The brine solution can be chilled by circulating to a remote refrigeration unit in a cooling loop, or cooled by piping containing refrigerant which is compressed remotely and passed through the vessel, as described in Nagoshi U.S. Pat. No. 4,601,909. The brine is preferably stirred by a stirring unit in the vessel, so that a desired uniform temperature is maintained.

Use of a brine solution having a controlled low temperature for freezing tissue samples is well known. In particular, a brine solution containing cruciferous oil has heat transfer properties which, when the brine is in a temperature range of −22° C. to −43° C., promote freezing at a rate that does not cause cellular damage. See, e.g., Liberman U.S. Pat. No. 4,840,035. In histological applications, it is sometimes desirable to achieve a uniform semi-frozen state where the tissue is firm enough to be sliced into very thin slices for testing and analysis. Here too it is desirable to have a controlled heat transfer rate. If it chills too fast, cells can freeze and rupture. If it chills to slowly, the tissue can deteriorate.

A research or clinical histology laboratory processes a large number of tissue samples for examination and it is important that the tissue samples be prepared as efficiently and safely as possible. After resection, samples should immediately be frozen to preserve and prepare for downstream studies that include sectioning, staining, labeling, immunological, RNA and DNA analysis and histological studies. GLP (Good Laboratory Practices) certifications require that samples have varying biosafety levels. GLP embodies a set of principles that provides a framework within which laboratory studies are planned, performed, monitored, recorded, reported and archived. These studies are undertaken to generate data by which the hazards and risks to users, consumers and third parties, including the environment, can be assessed for pharmaceuticals (only preclinical studies), agrochemicals, cosmetics, food additives, feed additives and contaminants, novel foods, biocides, detergents etc. GLP helps assure regulatory authorities that the data submitted are a true reflection of the results obtained during the study and can therefore be relied upon when making risk/safety assessments (The US FDA has rules for GLP in 21 CFR 58). A biosafety level is the level of the biocontainment precautions required to isolate dangerous biological agents in an enclosed facility. The levels of containment range from the lowest biosafety level 1 to the highest at level 4.

### SUMMARY OF THE INVENTION

A treatment system according to the invention is designed to solve the problems outlined in these requirements. To prevent health hazards due to exposure of contaminated samples and reagents (principally brine and cleaning solvents), the system incorporates a replaceable brine cartridge having a sealable outlet that isolates the fresh brine. This also functions to prevent undesired amounts of evaporation and recognizes the brine's hydroscopic uptake of atmospheric water that can cause brine imbalance. The use of a replacement cartridge reduces the contamination of the bath caused by processing multiple samples in a common bath. It likewise ensures the integrity of the brine.

The replacement cartridge includes a container having an inlet and an outlet, a first compartment communicating with the inlet, and a second compartment communicating with the outlet, wherein the second compartment is collapsible. A first conduit connects the working vessel to the inlet, and a second conduit is connected to the outlet for providing fresh fluid to the working vessel. When it becomes necessary to change the fluid in the working vessel, used fluid can be pumped to the first compartment by a pump in the first conduit, and fresh fluid can be released from the second compartment as it collapses. Where the used fluid is a chilled brine solution, it will pre-chill the fresh brine solution in the cartridge.

According to a preferred embodiment, the fresh fluid is held in a holding tank in the second conduit while the working vessel and the refrigeration loop are cleaned. The use of a collapsible second compartment optimizes use of the internal volume of the cartridge, so that the cartridge can have a compact design.

The size of the working vessel, e.g. the treatment station where samples are chilled or frozen, depends to some extent on the mass of samples immersed in the bath. The bath should have a sufficient volume to absorb the latent heat of the samples without significantly impacting the temperature of the bath. The working fluid should therefore have a mass on the order of 40 times or greater that of the samples.

Where only a small working vessel is needed, the overall system can have a compact design and be designed to be mobile. Alternatively, the working vessel can be designed for rapid disconnect from the first conduit and the cooling loop, so that samples can be transported in the brine solution. In this case it is preferable to provide the vessel with well-insulated walls and a cover. Mobility of the system and/or the working vessel permits moving the samples to remote upstream or downstream processing locations.

### BRIEF DESCRIPTION OF THE DRAWINGS

FIG. 1A is schematic cross-sectional view of a first embodiment of the treatment system, with a holding tank for fresh brine; according to the invention;

FIG. 1B is a schematic cross-sectional view according to a second embodiment, wherein the brine replacement cartridge does not have a movable divider separating the compartments;

FIGS. 2A and 2B are cross-sectional schematic diagrams according to a third embodiment, wherein the brine solution replacement cartridge has a flexible divider;

FIG. 3 is a cross-sectional schematic diagram, according to a fourth embodiment, wherein the first compartment has a fixed volume;

FIG. 4 is a cross-sectional schematic diagram according to a fifth embodiment, wherein the first compartment is filled before the second compartment collapses; and

FIG. 5 is a schematic view of a sixth embodiment, with a holding tank for used brine.

### DETAILED DESCRIPTION OF THE PREFERRED EMBODIMENTS

The present invention involves a treatment system employing a replacement cartridge for quickly and easily replacing the working fluid in a working vessel, particularly a brine solution for chilling and/or freezing tissue samples.

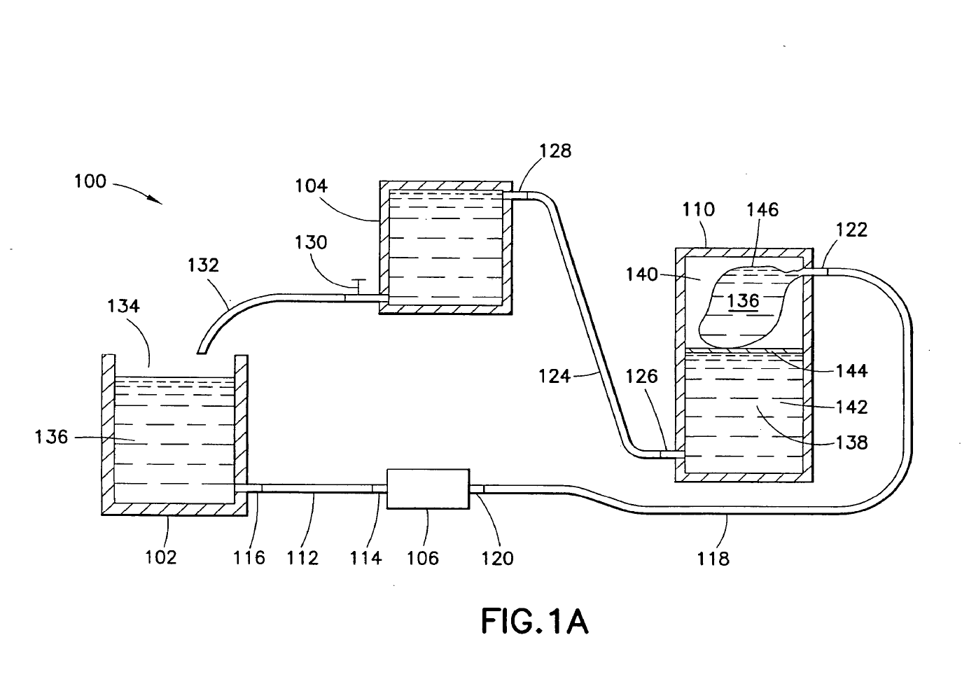

Referring to FIG. 1A, the treatment system 100 includes a tissue sample chilling vessel 102, a fresh brine holding vessel 104, a pump 106, and a brine solution replacement cartridge 110. The pump 106 is in fluid communication with chilling vessel 102 via hose 112, which is coupled to one-way valves 114 and 116. The pump 106 is also in fluid communication with brine solution replacement cartridge 110 via hose 118, which is coupled to one-way valves 120 and 122. As used herein, the term “one-way valve” will be understood to mean either a check valve or a pressure valve that opens at a threshold pressure differential. The brine solution replacement cartridge 110 is also in fluid communication with the fresh brine holding vessel 104 via hose 124, which is coupled to one-way valves 126 and 128. The holding vessel 104 also includes a manual outlet valve 130 in fluid communication with a hose 132. The hose 132 empties into the chilling vessel 102 through opening 134.

The brine solution replacement cartridge 110 includes a container having a first compartment 140 in which used brine is stored, and a second compartment 142 in which fresh brine is stored. The first compartment 140 houses a bladder 146, which is connected to the inlet having one-way valve 122. A moveable divider 144 separates compartment 140 from compartment 142. While only shown schematically, the divider 144 will preferably have the form of a piston carrying a seal riding against the inside wall of the container, which is preferably cylindrical.

Still referring to FIG. 1A, following sample treatment, the chilling vessel 102 holds used brine solution 136, and the brine solution replacement cartridge 110 holds fresh brine solution 138 in second compartment 142. To replace the used brine, a user engages the pump 106, which pumps the used brine solution 136 from chilling vessel 102 into the bladder 146. As the bladder 146 fills with used brine solution 136, it expands and pushes against the movable divider 144, which causes the second compartment 142 to collapse. This forces the fresh brine solution 138 through the one-way output valve 126, through the hose 124, through the one-way input valve 128, and into the holding vessel 104. The fresh brine solution 138 is stored in holding vessel 104 until the chilling vessel 102 is emptied and cleaned. After the chilling vessel 102 has been cleaned, a user actuates the one-way output valve 130, which allows the fresh brine solution 138 to flow through hose 132 into the chilling vessel 102. The brine solution will then be chilled by circulating to a remote refrigeration unit via a cooling loop. Alternatively, the brine solution can be chilled by refrigerant passing through coils immersed in the brine solution.

In another embodiment, illustrated in FIG. 1B, brine solution replacement cartridge 110 does not include a movable divider and the fresh brine solution 138 is forced out of the brine solution replacement cartridge 110 by the expanding bladder 146 alone. In this case the bladder itself forms the first compartment 140.

In still another embodiment, not illustrated, the system 100 does not include the pump 106, and the old brine solution 136 is forced into the bladder 146 via gravity, i.e., the brine solution replacement cartridge 110 is positioned below the chilling vessel 102.

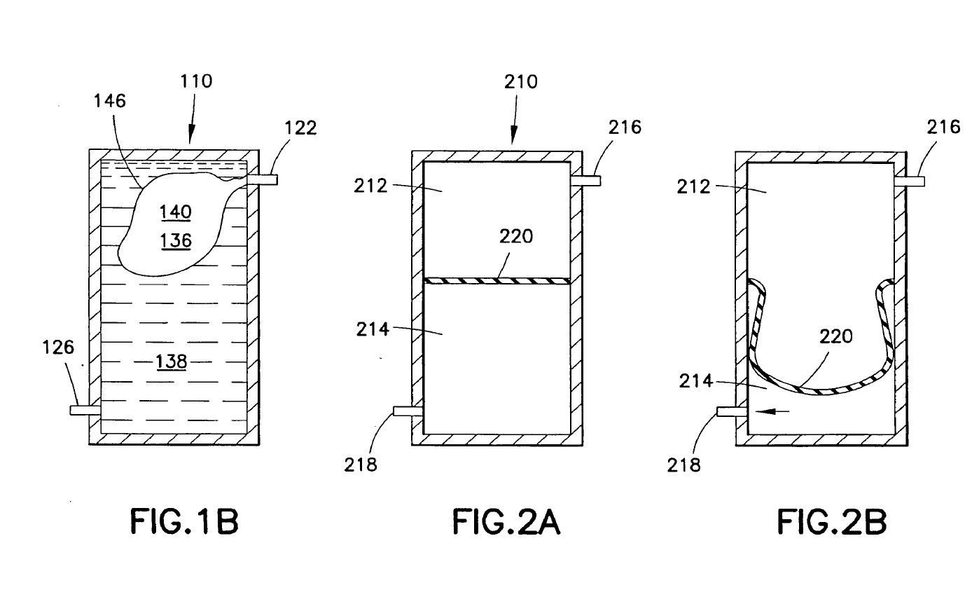

Referring to FIGS. 2A and 2B, the brine solution replacement cartridge 210 includes a first compartment 212 for used brine and a second compartment 214 for fresh brine. An inlet with one-way valve 216 is in fluid communication with the first compartment 212, and an outlet with one-way valve 218 is in fluid communication with the second compartment 214. The first compartment 212 is separated and sealed from the second compartment 214 by an expandable membrane 220. When the used brine solution is pumped into the first compartment 212 through one-way input valve 216, the membrane 220 expands to force the fresh brine solution 138 out of second compartment 214 through the one-way output valve 218 and toward the holding vessel 104 (FIG. 1A).

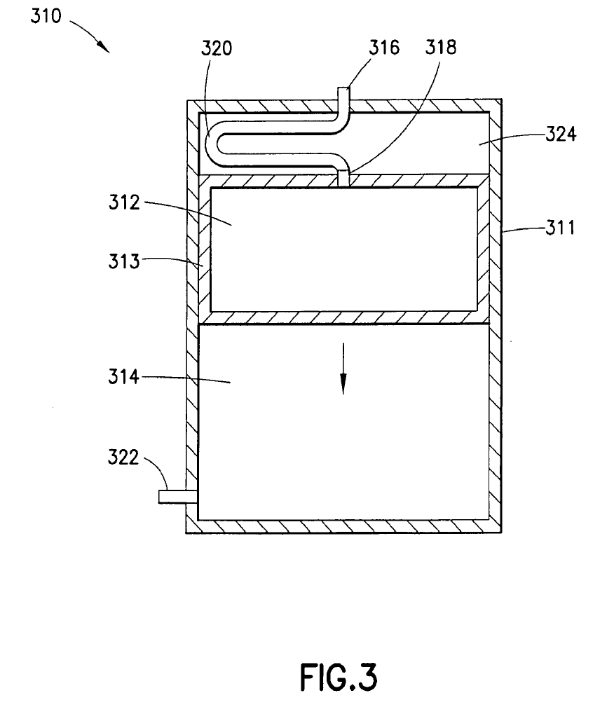

Referring to FIG. 3, the brine solution replacement cartridge 310 includes a first compartment 312 formed by a hollow piston 313 which is movable inside the container 311, and a second compartment 314 which collapses as the piston 313 moves downward, thereby expelling fresh brine. An inlet with a one-way valve 316 disposed in the wall of container 311 is in fluid communication with a one-way valve 318 in piston 313 via hose 320, whereby used brine can be pumped into first compartment 312. An outlet with one-way valve 322 is in fluid communication with the second compartment 314. When used brine solution is pumped into first compartment 312, it gains weight and causes the second compartment 314 to collapse, forcing out fresh brine. The hose 320 disposed in the space 324 is long enough to allow the piston 313 to move to the bottom of the compartment second 314. Note that this arrangement, like the already described embodiments, requires a holding vessel for the fresh brine.

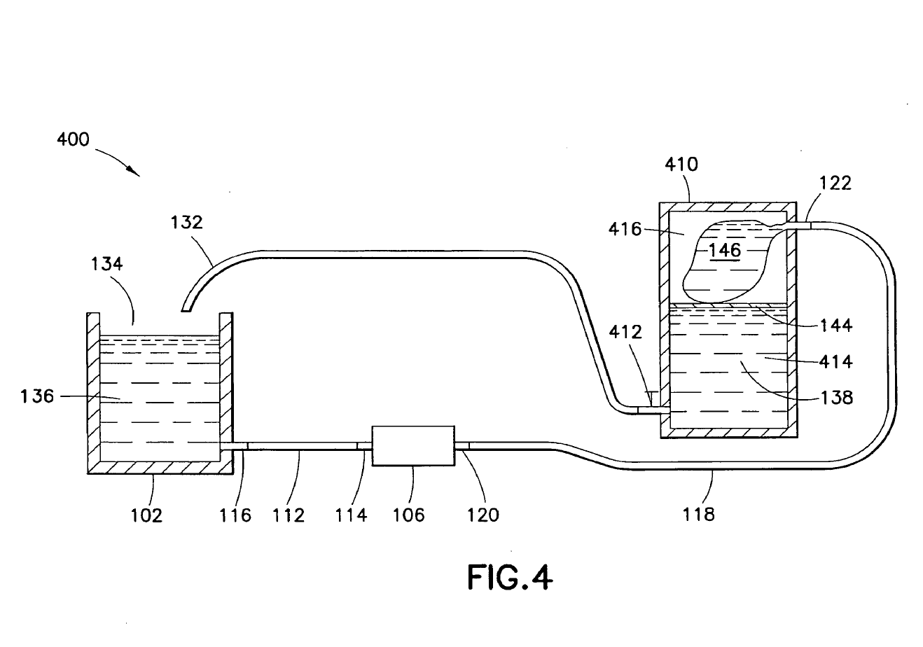

Referring to FIG. 4, another treatment system 400 includes a tissue sample chilling vessel 102, a pump 106, and a brine solution replacement cartridge 410. The inlet of pump 106 is connected to the chilling vessel 102 via hose 112, which is coupled to one-way valves 114 and 116. The output of pump 106 is connected to cartridge 410 via hose 118, which is coupled to one-way valves 120 and 122. The hoses 112 and 118 collectively form a first conduit, in which the pump is interposed. The cartridge 410 is connected to a second conduit formed by hose 132 via user controllable output valve 412. The hose 132 empties into the chilling vessel 102 through opening 134.

The brine solution replacement cartridge 410 includes first compartment 416 for used brine and a second compartment 414 for fresh brine. The first compartment 416 houses a bladder 146, which is connected to the one-way input valve 122. A movable divider 144 separates compartment 416 from compartment 414. The cartridge 410 is large enough to hold the used brine solution 136 and the fresh brine solution 138 at the same time.

Initially, the chilling vessel 102 holds used brine solution 136, and the brine solution replacement cartridge 110 holds fresh brine solution 138 in second compartment 414. In operation, a user engages the pump 106, which pumps used brine solution 136 from the chilling vessel 102 into bladder 146. The bladder 146 is allowed to fill completely with used brine solution 136. As the bladder 146 fills, it expands and weighs on the movable divider 144.

When the chilling vessel 102 is empty, it can be cleaned. Thereafter, a user opens valve 412 and the weight of the bladder 146 causes the second compartment 414 to collapse, forcing the fresh brine solution 138 through the one-way valve 412, though hose 132, and into chilling vessel 102.

The brine solution replacement cartridge 410 can be replaced with earlier described replacement cartridges, the only difference being that the cartridges be able to hold both used brine solution and fresh brine solution 138 at the same time, whereby the fresh brine solution can be dispensed at a later time and a holding vessel is not needed in the second conduit.

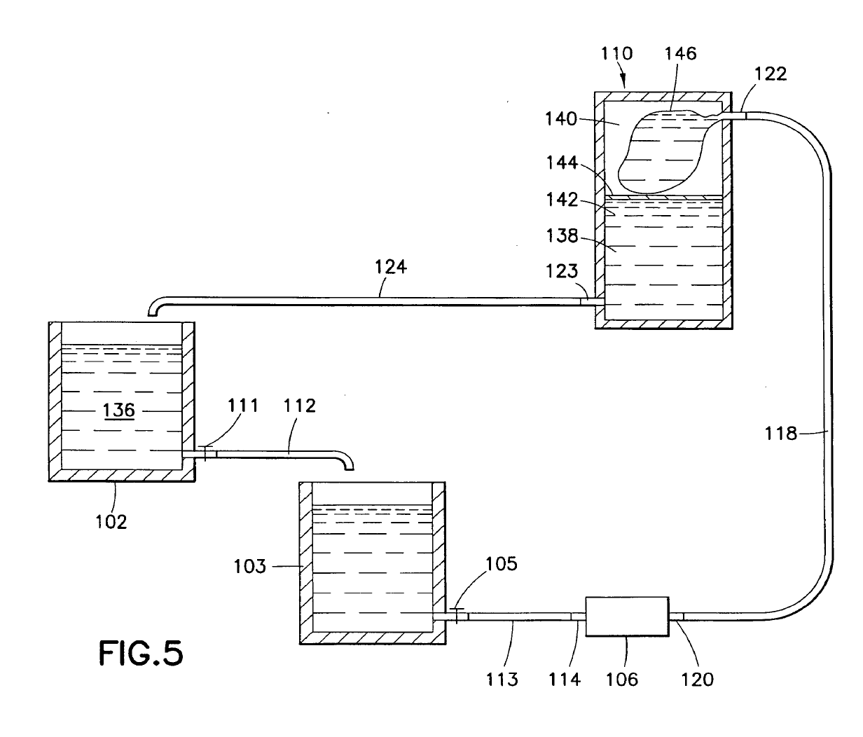

Referring to FIG. 5, a used brine holding vessel 103 is disposed in the first conduit section 112, 113. In this embodiment, the used brine solution 136 is drained from the chilling vessel 102 into the holding vessel 103 by opening valve 111. At this point the vessel 102 can be cleaned. The valve 105 is then opened, and the mixture is pumped by pump 106 to the first compartment 140 via first conduit section 118, and fresh brine solution is dispensed into the chilling vessel 102 via one-way valve 123 and second conduit 124. One-way valves 114, 120 are provided at the inlet and outlet of the pump, and one-way way valves 122, 123 are provided at the inlet and outlet of the replacement cartridge 110.

It is also possible to drain cleaning agents such as alcohol into the holding vessel 103, thereby forming a mixture with the used brine solution. In order to accommodate the mixture in the first compartment 140, this variation requires a replacement cartridge having a total capacity that is larger than the volume of fresh brine solution in the second compartment.

Because the old brine solution and alcohol cleaning solution have different boiling points, the alcohol solution is easily separated from the old brine solution when the old brine solution is recycled for re-use via heating methods.

Where the chilling vessel holds a volume of used brine solution that is greater than the storage capacity of a single brine solution replacement cartridge, two or more replacement cartridges can be used to empty the used brine solution from the chilling vessel, and then later refill the vessel with fresh brine solution. For example, if the chilling vessel holds three times the volume of a single replacement cartridge, three cartridges would be used to empty, and then later refill, the chilling vessel. A user would connect the first replacement cartridge as described above and empty one third of the used brine solution from the chilling vessel, then disconnect the first replacement cartridge. The user then repeats this process two more times to empty the second and third portions of the used brine solution from the chilling vessel. Thereafter, the chilling vessel is cleaned. Next, the first replacement cartridge is reconnected, and the fresh brine solution is dispensed into the chilling vessel as previously described above. The user then repeats this process two more times to dispense the second and third portions of the fresh brine solution into the chilling vessel. It is also possible to have manifolds on the inlet and outlet conduits, for connecting to multiple cartridges simultaneously.

Variations using a holding vessel in either the first or second conduit are possible. In the former case, all of the used brine solution is pumped into the holding vessel, the chilling vessel is cleaned, and the used brine solution is pumped sequentially into the first compartment of the three brine cartridges as the fresh brine solution is dispensed sequentially from the second compartments. In the latter case, used brine solution is pumped sequentially into the first compartments as fresh brine solution is dispensed sequentially into the holding vessel. The chilling vessel can then be cleaned, followed by filling with fresh brine from the holding vessel.

In every case, it is possible to design the cartridge so that the maximum capacity of the first compartment exceeds that of the second compartment, in order to contain cleaning solvents mixed with the used brine solution.

Variations, modifications, and other implementations of what is described herein may occur to those of ordinary skill in the art without departing from the spirit and scope of the invention. Accordingly, the invention is not to be defined only by the preceding illustrative description.

1. A treatment system comprising:
a working vessel for containing a working fluid wherein samples can be treated;
a replacement cartridge comprising a container having an inlet and an outlet, a first compartment communicating with the inlet, and a second compartment communicating with the outlet, wherein the second compartment is collapsible;
a first conduit connecting the working vessel to the inlet; and
a second conduit connecting the outlet to the working vessel;
whereby, used working fluid can be moved from the working vessel to the first compartment via the first conduit, and fresh working fluid in the second compartment can be forced into the second conduit as the second compartment collapses.

2. The treatment system of claim 1 further comprising a divider separating the first compartment from the second compartment, the divider being movable in the container, whereby the second compartment collapses as the first compartment expands.

3. The treatment system of claim 2 further comprising a bladder in the first compartment, the bladder being connected to the inlet and arranged to bear against the movable divider as the bladder is filled with used working fluid.

4. The treatment system of claim 3 further comprising a positive closing valve in the outlet, whereby the bladder can be completely filled with used working fluid before the valve is opened so that the second compartment can collapse.

5. The treatment system of claim 2 wherein the container is formed as a cylinder having an inside wall, and the divider is formed as a piston sealed against the inside wall.

6. The treatment system of claim 1 further comprising a bladder in the container, the bladder being connected to the inlet and forming the first compartment.

7. The treatment system of claim 1 further comprising a pump in the first conduit, the pump being arranged to pump used working fluid from the working vessel to the inlet of the first compartment.

8. The treatment system of claim 7 further comprising at least one one-way valve between the working vessel and the pump, and at least one one-way valve between the pump and the inlet.

9. The treatment system of claim 8 wherein at least one of said one-way valves is a pressure-controlled valve.

10. The treatment system of claim 1 further comprising a holding vessel in the second conduit, the holding vessel being arranged to hold fresh working fluid from the second compartment prior to releasing the fresh working fluid to the working vessel, whereby the working vessel can be cleaned after used working fluid has been moved to the first compartment and the second compartment has collapsed.

11. The treatment system of claim 10 further comprising at least one one-way valve between the outlet and the holding vessel.

12. The treatment system of claim 10 further comprising at least one positively closing valve between the holding vessel and the working vessel.

13. The treatment system of claim 1 further comprising a flexible membrane separating the first compartment from the second compartment, whereby the second compartment collapses as the first compartment expands.

14. The treatment system of claim 1 wherein the first compartment is formed by a hollow piston connected to the inlet by a hose, and wherein the second compartment collapses as the hollow piston moves in the container.

15. The treatment system of claim 14 wherein the hollow piston is arranged so that the second compartment collapses under the weight of the hollow piston as the hollow piston fills with used working fluid.

16. The treatment system of claim 14 further comprising at least one one-way valve between the inlet and the hollow piston.

17. The treatment system of claim 1 wherein the second compartment of the replacement cartridge is filled with a brine solution used for freezing tissue samples in the working vessel.

18. The treatment system of claim 1 further comprising a holding vessel in the first conduit, the holding vessel being arranged to hold used working fluid from the working vessel prior to moving the used working fluid to the first compartment, whereby the working vessel can be cleaned collapsing the second compartment and forcing fresh working fluid into the second conduit.

19. The treatment system of claim 1 wherein the first and second compartments each have a maximum volume, the maximum volume of the first compartment exceeding the maximum volume of the second compartment, whereby the first compartment can contain cleaning solvent mixed with used working fluid from the working vessel.
